(19) United States
(12) Patent Application Publication
(10) Pub. No.: US 2018/0267010 A1
(43) Pub. Date: Sep. 20, 2018

(54) WATER CUT AND PRESSURE SENSING DEVICE

(71) Applicant: General Electric Company, Schenectady, NY (US)

(72) Inventors: Nicholas J. Ellson, Leicester (GB); Frode Meringdal, Hagan (NO)

(21) Appl. No.: 15/925,097

(22) Filed: Mar. 19, 2018

Related U.S. Application Data
(60) Provisional application No. 62/473,556, filed on Mar. 20, 2017.

(51) Int. Cl. G01N 33/28; G01F 1/74; E21B 47/06; G01N 22/00

(52) U.S. Cl. CPC G01N 33/2847; G01N 33/2823; G01N 22/00; E21B 47/065; G01F 1/74

(57) ABSTRACT

Various embodiments of sensing devices are provided for determining water cut measurements of a fluid flowing through a fluid passageway. In some embodiments, the sensing device can include one or more pressure and/or temperature sensor probe assemblies configured to measure a pressure and/or temperature of the fluid, and a probe assembly configured to measure a water cut of the fluid.

### CROSS-REFERENCE TO RELATED APPLICATIONS

This application claims the benefit of U.S. Provisional Patent Application No. 62/473,556, filed Mar. 20, 2017, entitled “Subsea Water-Cut Sensor and Pressure Transducer,” the entirety of which is incorporated by reference.

### BACKGROUND

Hydrocarbons, such as petroleum and natural gas, can be extracted from underground reservoirs for use in energy production. In some cases, water can also be extracted concurrently with hydrocarbons, referred to as produced water. The volume ratio of the produced water to all liquids extracted is referred to water cut.

It can be desirable to measure water cut in a hydrocarbon extraction operation. In one aspect, understanding the amount of produced water on a well-by-well basis can facilitate reservoir engineering and improving recovery factor, a measure of the recoverable amount of hydrocarbons from a reservoir. In another aspect, because hydrates can form and restrict or block flow of extracted fluids when light hydrocarbons and water are present at certain pressures and temperatures, water cut measurements can be used for chemical dosing to manage hydrate formation and provide flow assurance. Water cut can also be measured at various transport points within a fluid network, such as to and from pipelines and tankers.

### SUMMARY

Instruments have been developed to measure water cut in surface and subsea environments. In one aspect, water cut can be measured by near-infrared (NIR) absorption spectroscopy. Produced water can be distinguished from other fluids extracted from a well by measuring absorption at selected NIR wavelengths. In another aspect, water cut can be measured by multiphase flow meters using ultrasound Doppler. Multiphase flow meters can contain a variety of sensors that are capable of determining water cut as well as flow rates of all of the parts (e.g., petroleum, natural gas, water, etc.) of the extracted fluid. However, each of these existing approaches can have drawbacks.

Water cut meters employing NIR absorption spectroscopy can be invasive, requiring placement of sensing equipment within the flow of extracted liquid (e.g., at about a center of a fluid carrying pipe). This positioning can disturb flow of the extracted liquids, as well as expose the sensing equipment to potential mechanical and/or chemical damage from the flow of extracted liquid.

Multiphase flow meters can be relatively large and complex, as well as expensive to purchase, install, and operate. The size requirements of a multiphase flow meter can be relatively large owing to the physical footprint of each sensor, which has its own sensing elements, housing, wiring. The complexity of multiphase meters can be relatively high due to the operation of multiple sensors, as well as the data, power, and installation requirements necessary for each sensor to operate properly. As a result, the number of multiphase flow meters employed in a given extraction site can be relatively limited, providing little redundancy of water cut measurement.

In general, systems and methods for measurement of water cut within a liquid, such as a flow of extracted liquid(s) from a well, are provided.

In one embodiment, a sensing device is provided and it can include a shaft, a pressure sensing assembly, a temperature sensor, and a near-field microwave probe assembly. The shaft can be generally elongate, extending along a longitudinal axis, and include a proximal portion and a distal portion. The shaft can also include a nose positioned at a terminal end of the distal shaft portion, opposite the proximal portion. The nose can define a chamber therein configured to receive a fluid from an environment external to the shaft. The pressure sensing assembly can include a generally elongate pressure sensor that is positioned within the chamber and extends longitudinally with respect to the shaft. The pressure sensing assembly can be configured to generate a pressure signal containing data representative of a pressure of a fluid received within the chamber. The temperature sensor can be positioned within the chamber and it can be configured to generate a temperature signal containing data representative of a temperature of a fluid received within the chamber. The near-field microwave probe assembly can be configured to contact a fluid of the external environment, transmit an incident microwave signal into a fluid of the external environment, receive a return microwave signal from a fluid of the external environment, and generate a near-field signal containing data representing a difference in at least one electrical property between the incident and return microwave signals.

In another embodiment, the sensing device can include one or more processors configured to receive the pressure signal, the temperature signal, and the near-field signal and to determine a water cut of a fluid of the fluid environment based upon at least the near-field signal and the temperature signal.

In another embodiment, the pressure sensor can include a diaphragm having a deformable surface that extends longitudinally with respect to the shaft and is in hydraulic communication with a fluid received within the chamber.

In another embodiment, the nose can include at least one hole formed through a distal facing surface and dimensioned to allow flow of the fluid between the external environment and the chamber.

In another embodiment, the near-field microwave probe can include a center conductor, a conductive shield, a first insulator, and a second insulator. The center conductor can include a distal probe tip. The conductive shield can extend about the center conductor and longitudinally recessed from the distal tip. The first insulator can be interposed between the center conductor and the conductive shield. The second insulator can extend about the probe tip and it can be longitudinally offset from a terminal end of the probe tip. The second insulator and the probe tip can extend through at least a portion of one of the plurality of openings.

In another embodiment, the external environment can be a fluid passageway containing a flow of a fluid and a distal facing surface of the nose can be shaped to substantially match a curvature of an inner wall of the fluid passageway.

In another embodiment, the sensing device can include a flange mounted on the shaft between the proximal shaft portion and the distal shaft portion and configured to couple the shaft to the fluid passageway.

In another embodiment, a distal facing surface of the nose can be configured to be substantially flush with an inner wall of the fluid passageway when the shaft is coupled to the fluid passageway.

Methods for determining water cut of a fluid are also provided. In one embodiment, the method can include positioning a distal end of a shaft of a sensing device in fluid communication with a fluid environment. The method can also include receiving, within a chamber of a sensing device, a fluid from the fluid environment. The chamber can be defined by a nose positioned at the distal end of the shaft. The method can further include generating, by a temperature sensor in thermal communication with the chamber, a temperature signal including date representing a temperature of the fluid received within the chamber. The method can additionally include transmitting, by a near-field microwave probe extending through the chamber, an incident microwave signal into the fluid within the fluid environment. The method can also include receiving, by the near-field microwave probe, a return microwave signal in response to interaction of the incident microwave signal with the fluid within the fluid environment. The method can further include generating, by a near-field microwave probe assembly, a near-field signal including data representing a difference in at least one electrical property between the incident microwave signal and the return microwave signal.

In another embodiment, the method can include determining, by at least one processor in communication with the temperature sensor and the near-field microwave probe assembly, a water cut of the fluid based upon the near-field signal and the temperature signal.

In another embodiment, the method can include generating, by a pressure probe assembly having a pressure sensor positioned within the chamber, a pressure signal including data representing a pressure of the fluid exerted upon the pressure sensor.

In another embodiment, the pressure sensor can include a diaphragm having a deformable surface that extends longitudinally with respect to the shaft and is in hydraulic communication with the fluid received within the chamber

In another embodiment, the fluid environment can be a pipe containing a through-hole extending from the outer wall of the pipe to an inner wall of the pipe. Positioning the distal portion of the shaft can include inserting the distal portion of the shaft within the through-hole such that a distal facing surface of the nose is substantially flush with an inner wall of the pipe.

In another embodiment, positioning the distal portion of the shaft can include coupling the sensing device to an outer wall of the pipe by a flange mounted on the shaft between the distal shaft portion and a proximal shaft portion.

In another embodiment, a distal facing surface of the nose can be shaped to substantially match a curvature of the fluid passageway.

In another embodiment, the fluid can be received by the chamber through one or more openings formed in a distal facing surface of the nose.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

These and other features will be more readily understood from the following detailed description taken in conjunction with the accompanying drawings, in which:

FIG. 1A is a side perspective view of one exemplary embodiment of a sensing device for measuring various parameters of a fluid passing through a fluid passageway;

FIG. 1B is a cross-sectional side view of the sensing device of FIG. 1A;

FIG. 2 is a cross-sectional side view of a distal portion of the water cut probe assembly of FIG. 1B for measuring water cut;

FIG. 3 is a cross-sectional side view of the sensing device of FIG. 1A coupled to a fluid passageway in one exemplary manner; and

FIG. 4 is a flow chart illustrating one exemplary embodiment of a process for determining water cut measurements of a fluid.

It is noted that the drawings are not necessarily to scale. The drawings are intended to depict only typical aspects of the subject matter disclosed herein, and therefore should not be considered as limiting the scope of the disclosure. In the drawings, like numbering represents like elements between the drawings.

### DETAILED DESCRIPTION

Water cut can refer to the amount of water within a flow of liquid extracted from a well (e.g., an oil well). Water cut measurements can be used in a variety of ways to increase the amount of oil recovered from an oil reservoir. As an example, water cut can provide information regarding the state of the oil reservoir, allowing an operator to adjust extraction parameters in response to changing reservoir conditions. Thus, it can be desirable to measure water cut in oil extraction operations. One current technology for measuring water cut, multiphase flow meters, can require multiple devices and, as a result, can be relatively large, expensive, and complex to maintain. Another current technology for measuring water cut, based upon absorbance of light (e.g., light in the near infrared wavelength region), can require placement of sensors deep within the flow of extracted fluids. This placement can disturb the fluid flow, as well as subject the sensors to mechanical and chemical damage. As discussed in detail below, improved devices, systems, and methods are disclosed that allow multiple parameters of a fluid to be measured using a single device. In one exemplary embodiment, a sensing device can measure electrical properties in addition to temperature and/or pressure of a fluid (e.g., oil) flowing through a fluid passageway (e.g., a tube or pipe). The use of a single device for measuring multiple parameters can reduce costs, simplify installation, and reduce repair requirements and repair costs. The sensing device can also measure the multiple fluid parameters in a manner that does not substantially interfere with fluid flow or subject the sensing device to unnecessary mechanical damage and chemical attack from the fluid flow.

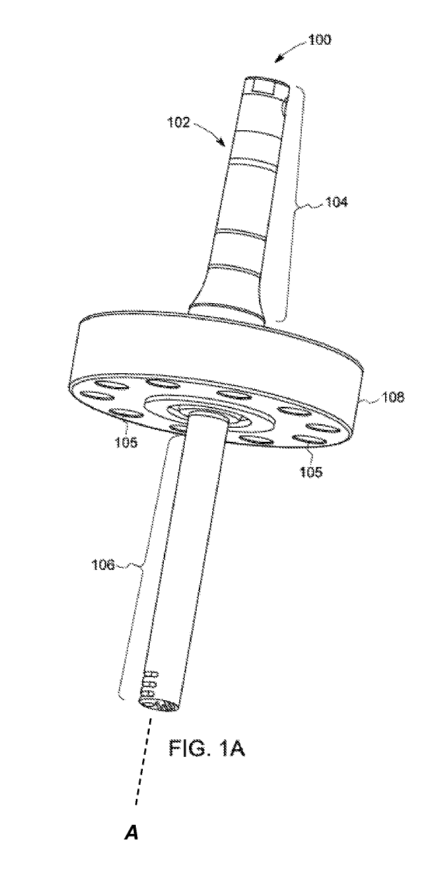

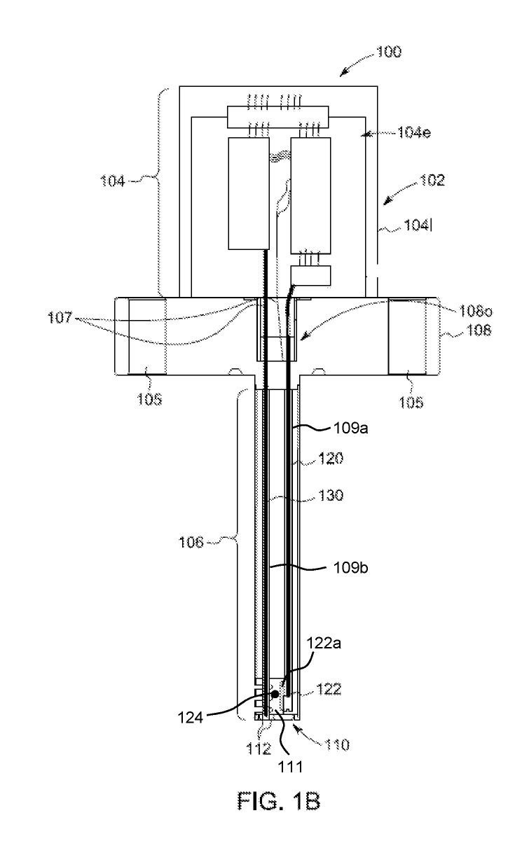

FIGS. 1A-1B illustrate one exemplary embodiment of a sensing device 100 that is configured to measure various parameters of a fluid passing trough a fluid passageway, such as a pipe, having the sensing device 100 coupled thereto. As shown, the sensing device 100 can include an elongate shaft 102 having a proximal portion 104 and a distal portion 106 extending along a longitudinal axis A. A main flange 108 can be mounted on the elongate shaft 102 between the proximal and distal portions 104, 106. The main flange 108 can be configured to facilitate mounting of the sensing device 100 (e.g., the elongate shaft 102) to a fluid passageway (e.g., a pipe) using fasteners such as screws or bolts passed through apertures 105 in the main flange 108.

The elongate shaft 102 of the sensing device 100 can have a variety of configurations. For example, in one embodiment, the elongate shaft 102 can have a cylindrical shape. Other configurations of the elongate shaft 102 are possible, such as different cross-sectional shapes (e.g., square, rectangular, triangular, hexagonal, octagonal) and longitudinal bends and/or curves. The elongate shaft 102 can be formed from a single housing, or the proximal and distal portions 104, 106 can be separate components that are mated to one another.

One or more probe assemblies for measuring various parameters of a fluid for use in determining water cut can be disposed within the elongate shaft 102. For example, the distal portion 106 can include a sensor probe assembly 120 having at least one sensor (e.g., temperature sensor, pressure sensor) for sensing at least one parameter associated with a fluid passing through a fluid passageway. The distal portion 106 can also include a water cut probe assembly 130 having a sensor configured to measure one or more electrical properties of the fluid.

The proximal portion 104 of the elongate shaft 102 can house various electronics 104e communicatively coupled to various probe assemblies, such as the sensor and water cut probe assemblies 120, 130. In one exemplary embodiment, as shown in FIG. 1B, the proximal portion 104 can include an inner housing 104i that is circumferential and extends through a central opening 108o in the main flange 108, and an outer housing (not shown) that can be disposed around the inner housing 104i for shielding the electronics 104e disposed therein. Various mating techniques can be used to mate the inner housing 104i and outer housing of the proximal portion 104 to the main flange 108, such as a sliding fit, threaded engagement, adhesive bonding, and/or snap-fit, and/or welding.

The electronics 104e can be configured to collect and process data received from the various probe assemblies, such as the sensor and water cut probe assemblies 120, 130. The electronics 104e can employ at least one of the pressure and temperature measurements in combination with the electrical property measurements for determination of water cut of the fluid. As shown, one or more wires 107 can extend through the proximal portion 104 and through the main flange 108 for coupling to the various probe assemblies. However, in alternative embodiments, not shown, the electronics 104e can wirelessly communicate with the probe assemblies. In further embodiments, the electronics can be located elsewhere in the sensing device, or even external to the sensing device.

The distal portion 106 can include a nose 110 at a terminal end, opposite the proximal portion. In one exemplary embodiment, at least a portion of the nose 110 can be hollow, defining a chamber 111 and one or more openings 112 formed therein for allowing fluid to flow into the nose 110, thereby allowing various parameters of the fluid to be sensed. The nose 110 can also have various shapes and be made out of one or more materials (e.g., conductive and/or non-conductive materials). For example, the nose 110 can be shaped to substantially match a curvature of the fluid passageway (e.g., concave). It can be appreciated that the sensing device 100 can have a variety of configurations, and can include any number of components mated in various ways depending on the intended use.

As indicated above, the distal portion 106 of the sensing device 100 can include any number of probe assemblies disposed therein, such as the sensor and water cut probe assemblies 120, 130. As shown in FIG. 1B, in one embodiment, the sensor probe assembly 120 can be in the form of a pressure probe assembly. In one embodiment, the pressure sensor 122 can be in the form of a diaphragm pressure sensor and include a diaphragm 122a having a deformable surface that extends longitudinally with respect to the shaft 102. One side of the deformable surface can be in hydraulic communication with fluid received within the chamber 111. An opposing side of the deformable surface can be in hydraulic communication with a with a transmission fluid (e.g., a substantially incompressible fluid). The transmission fluid can be contained within a first channel 109a extending along the distal portion 106, between the main flange 108 and the pressure sensor 122. A pressure sensing element (not shown) can be positioned in the proximal portion 104, in hydraulic communication with the diaphragm 122a via the transmission fluid, and can receive pressure exerted upon the diaphragm 122a via the transmission fluid. In response to pressure transmitted by the transmission fluid, the pressure sensing element can generate a pressure signal containing data representative of the sensed fluid pressure. The pressure signal can be received by the electronics 104e for processing.

In one exemplary embodiment, as shown in FIG. 1B, the diaphragm 122a can extend longitudinally with respect to the elongate shaft 102 (e.g., substantially parallel to the length of the elongate shaft 102), although other configurations are possible. Such a configuration can occupy a smaller cross-sectional area of the distal portion 106 of the elongate shaft 102, thereby allowing additional probe assemblies to be disposed within the elongate shaft 102 while maintaining a desired shaft diameter.

The nose 110 of the distal portion 106 can be configured for fluid communication with a fluid environment external to the elongate shaft 102. As shown, the nose 110 can include one or more openings 112 formed therein (e.g., within a distal facing surface) that are configured to allow fluid flowing through a fluid passageway to enter the chamber 111 and contact the diaphragm 122a. Thus, when the nose 110 of the distal portion 106 is positioned in contact with fluid passing through a fluid passageway, the diaphragm 122a can respond to pressure changes in the fluid (e.g., deform). This deformation can transmit pressure across the diaphragm 122a to the transmission fluid, which can communicate pressure changes of the fluid within the fluid passageway to the pressure sensing element. The pressure sensing element can generate and transmit the pressure signal to the electronics 104e in the proximal portion 104 of the elongate shaft 102 for processing. The pressure data can be transmitted from the pressure sensing element to the electronics 104e through a wire 107 or it can be transmitted wirelessly.

It can be understood that, while a diaphragm-based pressure probe assembly 120 is discussed above, alternative embodiments of the sensing device can include any number of, other types of pressure sensors without limit. Examples can include, but are not limited to, piezo-resistive, resonating crystal, capacitive, and/or electromagnetic pressure sensors.

As noted above, the sensing device 100 can also or alternatively be configured to measure a temperature of a fluid flowing through a fluid passageway. In general, a temperature sensor 124 can be positioned within the nose 110 of the elongate shaft 102 (e.g., within the chamber 111) for sensing a temperature of a fluid flowing into or adjacent the nose 110. The temperature sensor 124 can generate a temperature signal containing data representing the measured temperature of the fluid and transmit the temperature signal to the electronics 104e in the proximal portion of the elongate shaft 102 for processing. The temperature data can be transmitted from the temperature sensor to the electronics 104e through a wire 107 or it can be transmitted wirelessly. Any number of a variety of temperature sensors can be used, such as a thermocouple, without departing from the scope of this disclosure.

In one embodiment, the temperature sensor 124 can be part of a separate probe assembly extending through the elongate shaft 102. As an example, as part of a separate probe assembly, the temperature sensor 124 can be positioned within a separate channel (not shown) extending between the main flange 108 and the chamber 111, similar to the channel 109a. In an alternative embodiment, the temperature sensor can be integral with the sensor probe assembly 120 or water cut probe assembly 130.

The distal portion 106 of the elongate shaft 102 can also include a water cut probe assembly 130 that can be configured to measure a water cut of a fluid flowing through a fluid passageway. The water cut probe assembly 130 can extend within a second channel 109b that extends from the main flange 108, through the distal portion 106, and can terminate at the nose 110 of the sensing device 100 (e.g., within the chamber 111). As discussed in greater detail below, the water cut probe assembly 130 can be configured to contact a fluid in a fluid passageway and to measure a difference in electrical properties between an incident electrical signal that is transmitted into the fluid from the water cut probe assembly 130 (e.g., an incident microwave signal) and a return signal that is received and sensed by the water cut probe assembly 130 (e.g., a return microwave signal). For example, the water cut probe assembly 130 can assist with determining the contents of the fluid (e.g., water content) based in part on the difference in electrical properties (e.g., current, voltage, frequency, amplitude, phase, etc.) of transmitted and returned signals that are sent and received, respectively, by the water cut probe assembly 130.

The water cut probe assembly 130 can include one or more sensors (e.g., impedance sensor, permittivity sensor) that sense the electrical properties (e.g., current, voltage, resistance, capacitance, inductance, admittance, etc.) of the transmitted and returned electrical signals and send a near-field signal containing data representing a difference between the sensed electrical properties of the incident and return microwave signals to the electronics 104e for processing. For example, the electronics 104e can include a printed circuit board and/or processor having an algorithm that can determine (e.g., calculate) water cut of the fluid using such sensed electrical data, as well as the pressure and/or temperature measurements. In some embodiments, some of the electronics 104e and/or one or more sensors can be positioned in or adjacent to the nose 110. Systems and methods for detecting multi-phase fluid content are disclosed in more detail in U.S. Publication No. 2016/0131601 entitled “Systems and Methods to Measure Salinity of Multi-Phase Fluids,” which is hereby incorporated by reference herein in its entirety.

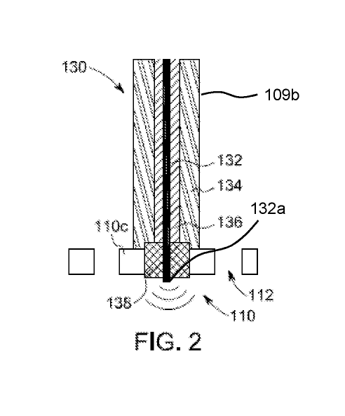

An expanded view of a portion of the nose 110 containing the water cut sensor assembly 130 is illustrated in FIG. 2. In one embodiment, the water cut probe assembly 130 can be in the form of a microwave near-field probe including a center conductor 132, a conductive shield 134, a first insulator 136, and a second insulator 138. The center conductor 132 can extend along the water cut probe assembly 130 and terminate at a probe tip 132a. The conductive shield 134 can be disposed around the center conductor 132 such that the center conductor 132 and the conductive shield 134 are substantially coaxial. The conductive shield 134 can extend along the distal portion 106 of the elongate shaft 102 and it can mate with a conductive material 110c that forms a part of the nose 110. This configuration can provide an electrically conductive pathway from the conductive material 110c of the nose 110 to the conductive shield 134.

The first insulator 136 can be positioned between the center conductor 132 and the conductive shield 134, along at least a portion of the length thereof, thereby preventing a direct electrical pathway between the center conductor 132, the probe tip 132a and the conductive shield 134. As shown in FIG. 2, the conductive shield 134 can be longitudinally recessed from the probe tip 132a and the second insulator 138 (e.g., glass) can be positioned at the distal-most end of the water cut probe assembly 130 (e.g., surrounding at least a portion of the probe tip 132a to provide separation between the probe tip 132a and any conductive material that forms the nose 110 of the sensing device 100. In this manner, the probe tip 132a can be coupled to the nose 110 and can extend through at least a portion of one of the openings 112 for contact with the fluid flowing through the fluid passageway. In certain embodiments, the second insulator 138 can be longitudinally recessed from a terminal end of the probe tip 132a. That is, at least a portion of the terminal end of the probe tip 132a can extend outside of the second insulator 138. It can be appreciated that the first and second insulators 136, 138 can be formed from any one of a variety of insulating materials, such as plastic, PVC, polyethylene, Teflon®, ceramic, glass materials, and the like.

In use, an electrical signal can be transmitted along the region between the center conductor 132 and the conductive shield 134 and into a fluid in contact with the center conductor 132. A portion 140 of the transmitted electrical signal can travel into the fluid and a portion of the signal can be reflected or returned back along the region between the center conductor 132 and the conductive shield 134. The return signal can travel along the region between the center conductor 132 and the conductive shield 134 to be sensed by at least one sensor (e.g., voltage sensor, current sensor, frequency sensor, amplitude sensor) of the electronics 104e in the proximal portion 104 of the sensing device 100. The electronics 104e can also measure a reflection coefficient of the return signal, and then use the reflection coefficient to determine a permittivity of the fluid. The water cut of the fluid can be found from the permittivity using permittivity equations. In some embodiments, the electronics 104e can include a printed circuit board configured to analyze the sensed data, including the sensed data associated with the return signal, such as for determining various characteristics of the fluid. For example, the return signal can be analyzed to determine an impedance, voltage, admittance, inductance, and/or capacitance of the fluid. Other characteristics of the fluid can also be determined.

The distal end of the water cut probe assembly 130 can have a variety of other configurations. For example, the second insulator 138 of the nose 110 can include a variety of shapes and configurations. In some implementations, for example, the second insulator 138 can have a convex shape that extends distally from the nose 110 and/or the second insulator 138 can cover a distal end of the center conductor 132.

Salinity can affect the permittivity of water, which can affect the mixture permittivity. Water permittivity can be used in mixture models along with mixture permittivity to estimate water cut. For accuracy of water cut estimates, it can be necessary to account for salinity into water permittivity. For example, a method for estimating water cut can include first estimating a mixture or liquid permittivity using measured admittance. Salinity of water can then be estimated using admittance or permittivity. Then complex permittivity of water can be estimated using a Stogryn model with temperature and salinity as inputs at the frequency of operation. Mixture permittivity and water salinity can then be fed into at least one mixture model to estimate water fraction and then water cut (e.g., using mixture density or gas fraction).

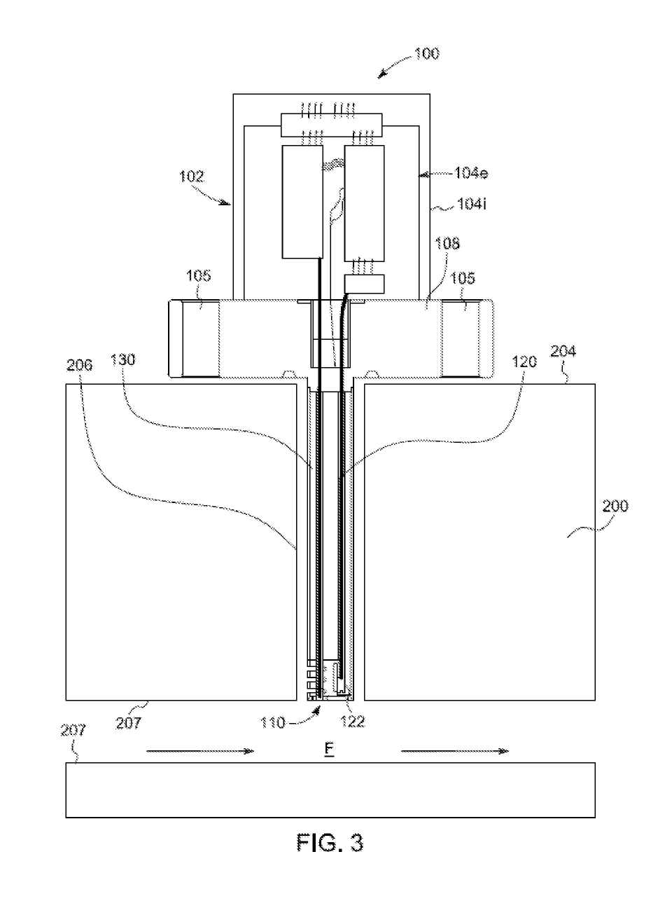

FIG. 3 illustrates the sensing device 100 of FIGS. 1A-1B coupled to a fluid passageway 200 in an exemplary configuration. The fluid passageway 200 can be, for example, a pipe. As indicated above, the sensing device 100 can include a main flange 108 mounted on the elongate shaft 102. The main flange 108 can have any shape (e.g., a cylindrical shape as shown) that allows the main flange 108 to rest on an outer wall 204 of the fluid passageway 200 so as to allow the distal portion 106 of the elongate shaft 102 to extend near or into a fluid passageway 200. As shown in FIG. 3, the main flange 108 can include one or more coupling features, such as mounting holes 105, extending therethrough. The coupling features can be disposed around the main flange 108 and can extend entirely through the main flange 108 for receiving a fastener or other mating device to mate the sensing device 100 to a fluid passageway 200, as shown. Furthermore, although a main flange 108 having mounting holes 105 are shown for assisting with coupling the sensing device 100 to a fluid passageway 200, the sensing device 100 can include any number of features and configurations that allow the sensing device 100 to be coupled to a fluid passageway. For example, the sensing device can include an elongate shaft 102 but not a main flange 108. In this configuration, for example, the elongate shaft 102 can be directly screwed, welded, and/or clamped to the fluid passageway.

With the main flange 108 positioned against the outer wall 204 of the fluid passageway, the distal portion 106 of the elongate shaft 102 can extend into a through-hole 206 formed through fluid passageway 200. As a result, the nose 110 of the sensing device 100 can be positioned in contact with the fluid F flowing through the fluid passageway 200. The sensing device 100 can thereby sense and collect data associated with at least one parameter of the fluid (e.g., electrical properties, pressure, and/or temperature) for determination of water cut. The nose 110 can be positioned substantially flush with an inner wall 207 of the fluid passageway 200 (e.g., as shown in FIG. 3), or radially outward/inward of the inner wall 207 of the fluid passageway 200.

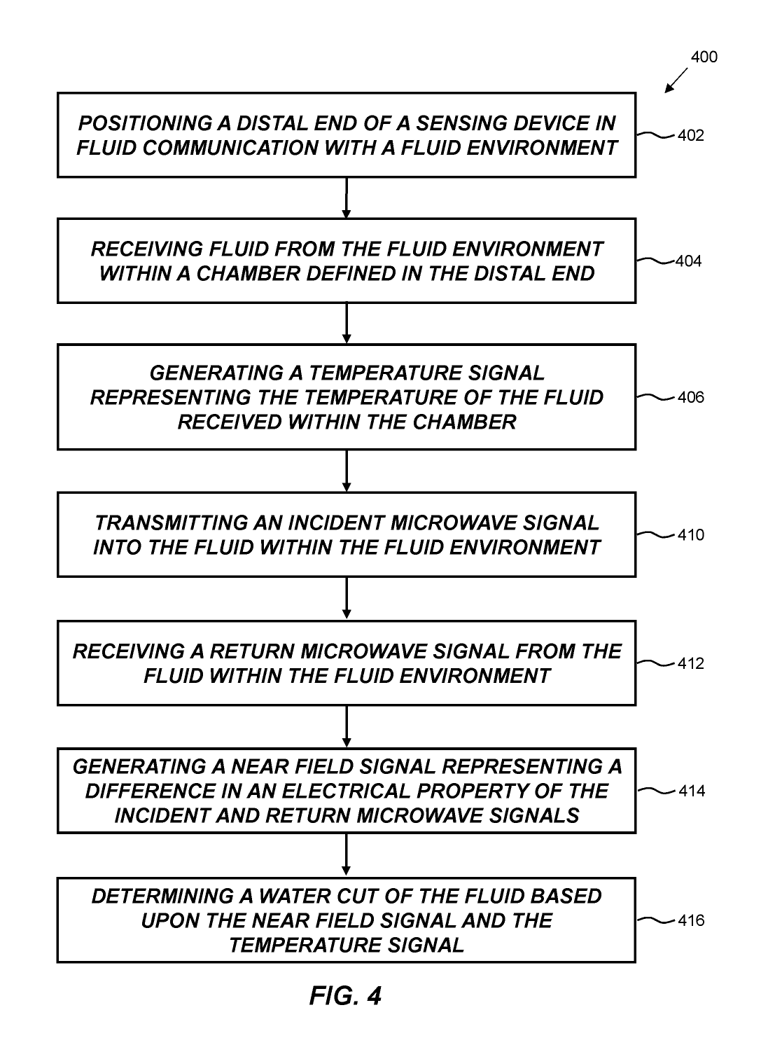

FIG. 4 is a flow diagram illustrating an exemplary embodiment of a method 400 for determining water cut of a fluid. The method 400 is discussed in with reference to the sensing device 100. In certain aspects, embodiments of the method 400 can include greater or fewer operations than illustrated in FIG. 4 and the operations can be performed in a different order than illustrated in FIG. 4.

In operation 402, a distal end of the elongate shaft 102 of the sensing device 100 (e.g., the distal end of the distal portion 106) can be positioned in fluid communication with a fluid environment. The fluid environment can be the fluid F flowing through the fluid passageway 200 (e.g., a pipe). The distal portion 106 of the elongate shaft 102 can be inserted within a through hole extending between the outer wall 204 and the inner wall 207 of the fluid passageway 200. The elongate shaft 102 can be dimensioned such that a distal facing surface of the nose 110 is substantially flush with the inner wall 207 when the sensing device 100 is mounted to the fluid passageway 200. The sensing device 100 can be coupled to the fluid passageway 200 by the flange 108. The distal facing surface of the nose 110 can be shaped to substantially match a curvature of the inner wall 207 of the fluid passage 200. In this manner, a substantially smooth transition between the inner wall 207 and the nose 110 can be provided and disruption of the flow of fluid F can be due to the presence of the sensing device 100 can be reduced or substantially eliminated.

In operation 404, fluid from the fluid environment can be received within the chamber 111 within the distal end of the sensing device 100. The chamber 111 can be defined by the nose 110 positioned at the distal end of the distal portion 106 of the elongate shaft 102. As an example, the fluid F can flow between the chamber 111 and the fluid environment through the one or more openings 112 formed in the distal facing surface of the nose 110.

In operation 406, the temperature sensor 124 can generate a temperature signal representing the temperature of the fluid F received within the chamber 111. As an example, the temperature sensor 124 can be positioned in thermal communication within the chamber 111 (e.g., within or adjacent to the chamber 111).

In operation 410, an incident microwave signal can be transmitted into the fluid F within the fluid environment. In operation 412, a return microwave signal can be received from the fluid F within the fluid environment. The water cut probe assembly 130, including a near-field microwave probe assembly, can transmit and receive the incident and return microwave signals.

In operation 414, the water cut probe assembly 130 can generate a near-field signal based upon the incident microwave signal and the return microwave signal. The near-field signal can include data representing a difference in at least one electrical property of the incident and return microwave signals. Examples of electrical properties can include, but are not limited to, current, voltage, resistance, capacitance, inductance, and admittance.

In operation 416, water cut of the fluid F can be determined. As an example, a processor of the electronics 104e can determine the water cut based at least upon the near-field signal and the temperature signal.

Optionally, the sensing device 100 can further measure pressure of the fluid F. As an example, the pressure sensor 122 of the pressure probe assembly 120 can positioned within the chamber 111 for hydraulic communication with the fluid F. Thus, when the fluid F is received within the chamber 111, the fluid F can exert pressure upon the pressure sensor 122, causing the diaphragm 122a to deform. This deformation can transmit the pressure across the diaphragm 122a to the transmission fluid and the transmission fluid can in turn convey the pressure to the pressure sensing element. The pressure sensing element can generate and transmit the pressure signal to the electronics 104e for processing to determine the pressure exerted on the pressure sensor. The pressure sensor 122 can be configured such that the diaphragm 122a extends longitudinally with respect to the distal portion 106 of the elongate shaft 102. So configured, the lateral dimension occupied by the pressure sensor 122 can be minimized, providing space for accommodation of at least a portion of the water cut probe assembly 130 and the temperature sensor 124 within the chamber 111.

In some embodiments, the sensor and water cut probe assemblies 120, 130 can be configured to be disposed within a pre-existing housing of a pressure and/or temperature sensing device, thereby allowing the device to measure water cut in addition to pressure and/or temperature. This approach can also reduce labor and installation costs since the sensing device can be efficiently integrated into an existing system using existing coupling features.

Exemplary technical effect of the methods, systems, and devices described herein include, by way of non-limiting example, non-invasive measurement of electrical and physical properties of a fluid flowing in a fluid passageway for determination of water cut using a single sensing device. The cost of ownership and operation of the disclosed sensing devices can be significantly less than that of multiphase flow meters having comparable measurement capabilities. The complexity of the disclosed sensing devices can be significantly less than that of multiphase flow meters, providing improved reliability and reducing infrastructure costs associated with power, communication, and supporting structures. The volume of space occupied by the disclosed sensing devices can be less than multiphase flow meters, placing less constraints on system integrators and allowing for optimal placement for acquiring measurements from fluids. Redundancy of measurement can be provided, for both reliability and cross-correlation for liquid viscosity (e.g., in wet gas).

Certain exemplary embodiments will now be described to provide an overall understanding of the principles of the structure, function, manufacture, and use of the systems, devices, and methods disclosed herein. One or more examples of these embodiments are illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a standalone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. A sensing device, comprising:
a generally elongate shaft extending along a longitudinal axis that includes a proximal portion and a distal portion;
a nose positioned at a terminal end of the distal shaft portion, opposite the proximal portion, the nose defining a chamber therein configured to receive a fluid from an environment external to the shaft;
a pressure sensing assembly including a generally elongate pressure sensor that is positioned within the chamber and extends longitudinally with respect to the shaft, wherein the pressure sensing assembly is configured to generate a pressure signal containing data representative of a pressure of a fluid received within the chamber;
a temperature sensor positioned within the chamber and configured to generate a temperature signal containing data representative of a temperature of a fluid received within the chamber; and
a near-field microwave probe assembly configured to:
transmit an incident microwave signal into a fluid of the external environment;
receive a return microwave signal from a fluid of the external environment; and
generate a near-field signal containing data representing a difference in at least one electrical property between the incident and return microwave signals.

2. The sensing device of claim 1, further comprising one or more processors configured to receive the pressure signal, the temperature signal, and the near-field signal and to determine a water cut of a fluid of the fluid environment based upon at least the near-field signal and the temperature signal.

3. The sensing device of claim 1, wherein the pressure sensor comprises a diaphragm having a deformable surface that extends longitudinally with respect to the shaft and is in hydraulic communication with a fluid received within the chamber.

4. The sensing device of claim 1, wherein the nose includes at least one hole formed through a distal facing surface and dimensioned to allow flow of the fluid between the external environment and the chamber.

5. The sensing device of claim 4, wherein the near-field microwave probe comprises:
a center conductor including a distal probe tip;
a conductive shield extending about the center conductor and longitudinally recessed from the distal tip;
a first insulator interposed between the center conductor and the conductive shield; and
a second insulator extending about the probe tip and longitudinally offset from a terminal end;
wherein the second insulator and the probe tip extend through at least a portion of one of the plurality of openings.

6. The sensing device of claim 1, wherein the external environment is a fluid passageway containing a flow of a fluid and a distal facing surface of the nose is shaped to substantially match a curvature of an inner wall of the fluid passageway.

7. The sensing device of claim 6, further comprising a flange mounted on the shaft between the proximal shaft portion and the distal shaft portion and configured to couple the shaft to the fluid passageway.

8. The sensing device of claim, wherein a distal facing surface of the nose is configured to be substantially flush with an inner wall of the fluid passageway when the shaft is coupled to the fluid passageway.

9. A method for determining water cut of a fluid, comprising:
positioning a distal end of a shaft of a sensing device in fluid communication with a fluid environment;
receiving, within a chamber of a sensing device, a fluid from the fluid environment, wherein the chamber is defined by a nose positioned at the distal end of the shaft;
generating, by a temperature sensor in thermal communication with the chamber, a temperature signal including date representing a temperature of the fluid received within the chamber;
transmitting, by a near-field microwave probe extending through the chamber, an incident microwave signal into the fluid within the fluid environment;
receiving, by the near-field microwave probe, a return microwave signal in response to interaction of the incident microwave signal with the fluid within the fluid environment; and
generating, by the near-field microwave probe assembly, a near-field signal including data representing a difference in at least one electrical property between the incident microwave signal and the return microwave signal.

10. The method of claim 9, further comprising determining, by at least one processor in communication with the temperature sensor and the near-field microwave probe assembly, a water cut of the fluid based upon the near-field signal and the temperature signal.

11. The method of claim 9, further comprising generating, by a pressure probe assembly having a pressure sensor positioned within the chamber, a pressure signal including data representing a pressure of the fluid exerted upon the pressure sensor.

12. The method of claim 9, wherein the pressure sensor comprises a diaphragm having a deformable surface that extends longitudinally with respect to the shaft and is in hydraulic communication with the fluid received within the chamber

13. The method of claim 9, wherein the fluid environment is a pipe containing a through-hole extending from the outer wall of the pipe to an inner wall of the pipe and wherein positioning the distal portion of the shaft comprises inserting the distal portion of the shaft within the through hole such that a distal facing surface of the nose is substantially flush with an inner wall of the pipe.

14. The method of claim 13, wherein positioning the distal portion of the shaft further comprises coupling the sensing device to an outer wall of the pipe by a flange mounted on the shaft between the distal shaft portion and a proximal shaft portion.

15. The method of claim 13, wherein a distal facing surface of the nose is shaped to substantially match a curvature of an inner wall of the fluid passageway.

16. The method of claim 9, wherein the fluid is received by the chamber through one or more openings formed in a distal facing surface of the nose.
